(19) United States
(12) Patent Application Publication
(10) Pub. No.: US 2011/0251142 A1
(43) Pub. Date: Oct. 13, 2011

(54) USE OF TRITERPENIC AND ANTHRAQUINONE GLYCOSIDES AS SUBSTANCES EXERTING HYDRAGOGUE ACTIVITY ON MUCOSA OF UPPER RESPIRATORY APPARATUS FOR THE TREATMENT OF THE EFFECTS RESULTING FROM THE UPPER RESPIRATORY TRACT DISEASES

(75) Inventor: Sandro Soriano, Montella-Avellino (IT)

(73) Assignee: Sandro Soriano, Montella-Aversa (IT)

(21) Appl. No.: 13/051,336

(22) Filed: Mar. 18, 2011

(30) Foreign Application Priority Data
Sep. 23, 2008 (IT) RM2008A000506
Sep. 17, 2009 (IT) PCT/IT2009/000417

(51) Int. Cl. A61K 31/704; A61P 11/00; A61P 11/02; A61K 31/575

(52) U.S. Cl. 514/26; 514/180

(57) ABSTRACT

Use of triterpene and anthraquinone glycosides, as substances exerting hydragogue activity on mucosa of upper respiratory apparatus, for the treatment of the effects resulting from the upper respiratory tract diseases. The present invention concerns the use of triterpene and anthraquinone glycosides, as substances exerting hydragogue activity on mucosa of upper respiratory apparatus, for the treatment of the effects resulting from the upper respiratory tract diseases as for example, sinusitis, both acute or chronic, rhinosinusitis, rhinitis.

The present application is a continuation of PCT/IT2009/000417 filed on 17 Sep. 2009 which claims priority to Italian Patent Application No. RM2008A000506 filed on 23 Sep. 2008.

The present invention concerns the use of triterpene and anthraquinone glycosides as substances exerting hydragogue activity on the mucosa of upper respiratory apparatus for the treatment of the effects resulting from respiratory tract diseases. Particularly, the present invention concerns the use of triterpene and anthraquinone glycosides as substances exerting hydragogue activity on the mucosa of upper respiratory apparatus for the treatment of the effects resulting from respiratory tract diseases as, for example, mucosa edema of the upper respiratory tract, loss of the pneumatization of the paranasal sinuses, loss or reduction of the cilia motility, build-up of muco-purulent secretions in the paranasal sinuses.

Rhinosinusitis (the main disease of upper respiratory tract), consists of the mucosa surface inflammation of upper respiratory tract. The consequences of this pathology are represented mainly from the mucosa edema of upper respiratory tract, the loss of the normal pneumatization of the paranasal sinuses, loss or reduction of the cilia motility, build-up of muco-purulent secretions in paranasal sinuses. Such effects represent the main causes of painful symptomatology resulting from rhinosinusitis.

In sinusitis occurrence, the mucosa of inflamed sinuses swells and produces an excessive mucus amount. The mucosa swelling determines ostia narrowing resulting in disturbance of normal mucus drainage towards the nose and the mouth cavities. The mucus stagnating in great amount within the sinuses, constitutes a culture medium for bacteria, viruses or fungi which, from the nose or throat, can reach paranasal sinuses. In these cases the infection is added to the inflammation.

When the nasal mucosa swells it obstructs the ostium of the paranasal sinuses and oxygen occurring therein is adsorbed by mucosa blood vessels, the associated relative negative pressure resulting within the sinuses (ex vacuo sinusitis) provokes pain. If the vacuum persists, in the mucosa trasudate is formed filling up the sinus thus constituting a culture medium for bacteria entering the sinus through the ostium, or via a diffused cellulitis, or mucosa peculiar lamina thrombophlebitis. Serum and leukocyte exudate is formed in order to fight the infection and, in the obstructed sinus, a positive pressure is developed resulting in enhanced pain. The mucosa becomes hyperemic and edematous.

Where the blocking of the drainage ostium of the paranasal sinus is not completely eliminated, acute sinusitis tend to be chronic evolving in chronic form, leaving within the paranasal sinuses a remarkable muco-pus amount (rhinosinusitis effect resulting in total or partial loss of cilia motility (rhinosinusitis effect).

Up to now, the unique effective remedy in order to eliminate the muco-pus from the paranasal sinuses has been the surgical treatment, consisting of accurate cleaning of the involved sinus and creating a wide drainage pathway between sinus cavity and ipsilateral nasal fossa.

Accordingly it is therefore apparent the need to provide new methods and active principles suitable to overcome the disadvantages of prior art.

The author of the present invention has now found that, taking advantage of the hydragogue and surface activity of some substances, a process cascade is triggered resulting in the mucosa edema reduction of the nasal fossae, paranasal sinuses and the relative ostia resulting in pneumatization restoring of paranasal sinuses, as well as evacuation of muco-pus stagnating in the paranasal sinuses resulting in cilia motility restoring. As a result of this action, the physiological functions of paranasal sinuses and mucosa thereof are restored associated with disappearance of painful symptomatology derived from these effects.

In literature, there are several substances displaying hydragogue activity when in contact with human mucosa, but this activity never has been experimented and used on the mucosa of the nasal fossae, paranasal sinuses and relative ostia.

The substances that displayed a remarkable hydragogue activity on the mucosa of upper respiratory apparatus are represented from triterpene glycosides and anthraquinone glycosides, all naturally occurring as content of numerous plants, both herbaceous and arboreal.

Among triterpene glycosides that have demonstrated such hydragogue activity, there are: cucurbitacines, senegine and glycyrrhizine. These triterpene glycosides, as above noted, are contained in various plants, of which more common are: Citrullus colocynthis (Coloquintide), Brionya dioica (Brionia), Gratiola officinalis (Hedge Hyssop), Polygala Senega L. (Seneca snakeroot), Glycyrrhiza glabra (European licorice), Momordica (Siamese Momordica), Ipomoea purga (Jalap), Convulvulus scammonia (Scammony), Ecballium Elaterium (squirting cucumber).

Among anthraquinone glycosides, particularly, there are: emodins, chrysophanol, reine or physcion. These anthraquinone glycosides, as previously noted, are contained in various plants, of which more common are: Cassia Angustifolia (Senna), Rhamnus purshiana (Cascara Buckthorn), Rhamnus frangula (Frangula), Rheum palmatum (Rhubarb), Rumex crispus (Romice) and Aloe.

In order to demonstrate that the hydragogue and surface activity of said substances can be used successfully for the treatment of the effects resulting from sinusitis, in vivo studies on ciliate cells of the respiratory mucosa have been carried out by using isolated cucurbitacins B, D, I, E and aqueous extracts containing such substances, in particular Citrullus Colicyntis (Coloquintide) extract.

It has therefore been discovered that Cucurbitacines D, I, B and E (triterpenes) represent the main causative agents of hydragogue and surface activity.

Therefore it is a specific object of the present invention a pharmaceutical composition for the treatment of the effects resulting from sinusitis both acute and chronic, rhinosinusitis or rhinitis, said pharmaceutical composition being in a pharmaceutical form suitable to the nasal administration to the upper respiratory apparatus, for example aerosol, solution, spray, and consisting of at least one cucurbitacin or an extract containing thereof, together with one or more pharmaceutically acceptable adjuvant and/or excipients, said at least one cucurbitacin being present at concentration from 20 μg/ml to 120 μg/ml of total composition, preferably from 40 μg/ml to 60 μg/ml of total composition. The extract is preferably an aqueous extract, preferably an extract of Citrullus colocynthis (Coloquintide) or Ecballium Elaterium (squirting cucumber).

The effects resulting from the diseases of the upper respiratory tract are, for example, edema of upper respiratory apparatus mucosa, loss of the pneumatization of the paranasal sinuses, loss or reduction of the cilia motility, buildup of muco-purulent secretions in the paranasal sinuses.

The composition of the invention is able to produce hydragogue activity on the mucosa of the upper respiratory apparatus, increase the cilia beat in the ciliate cells and determine the evacuation of histamine from said mucosae. In fact, the topical administration of cucurbitacines to the upper respiratory apparatus at the dosage of the present invention is surprisingly able to induce local depolarization of epithelial cell membrane, reactivation and increasing of the cilia motion of ciliate cells. The depolarization activity involves all the epithelia mucosa cells of the upper respiratory tract and it is able to reach the cavity of paranasal sinuses. In this way, the elimination of the liquid (i.e. the secretion caused by the sinusitis) occurs also in sites distant from the point of contact with cucurbitacines. In addition, the reactivation and increasing of the cilia motility induce a rapid evacuation of mucu-purulent secretions that stagnate in paranasal sinus. The above mentioned activities allow the restoration of the normal pneumatization of the paranasal sinus (with the consequent disappearance of pain) and have an indirect anti-inflammatory effect.

Cucurbitacines can be selected from the group consisting of cucurbitacines D, I, B or E wherein the concentration of cucurbitacine D ranges from 7.5 μg/mL to 45.0 μg/mL, preferably from 15.5 μg/mL to 22.5 μg/mL, cucurbitacine I from 7.5 μg/mL to 45.0 μg/mL, preferably from 16.0 μg/mL to 23.0 μg/mL, cucurbitacine B from 4.5 μg/mL to 25 μg/mL, preferably from 7.6 μg/mL to 12.6 μg/mL, cucurbitacine E from 0.5 μg/mL to 5 μg/mL, preferably from 0.9 μg/mL to 1.9 μg/mL.

It is further object of the present invention is a pharmaceutical compositions as defined above for use in the treatment of the effects resulting from sinusitis both acute and chronic, rhinosinusitis or rhinitis, said effects being mucosa edema of the upper respiratory apparatus, loss of the pneumatization of the paranasal sinuses, loss or reduction of the cilia motility, build-up of muco-purulent secretions in paranasal sinuses.

Moreover, the medicament exerts surface action on muco-purulent secretions formed as a result of the diseases of the upper respiratory tract.

The present invention will be now described by an illustrative, but not limitative way, according to preferred embodiments thereof.

### EXAMPLE 1

### Study on Cucurbitacines D, I, B and E. as Main Causative Agent for Action Mechanism in the Treatment of the Effects Resulting from Sinusitis

In order to assess the responsibility and ability of cucurbitacines D, I, B and E, to perform an hydragogue action on nasal and paranasal mucosa, tests with aqueous solutions containing cucurbitacines at various concentrations have been carried out.

Extraction Method

The extraction of cucurbitacines has been carried out from Coloquintide fruit according to the following procedure: coloquintide fruits, collected at full maturation time, have been deprived of epicarpum and cut in little pieces. 200 grams of cut fruits have been sampled and placed in a 1000 ml beaker, to which 500 mm of bi-distilled water have been added. The mixture containing beaker (water+coloquintide fruits) has been placed on electric heating plate and heated at temperature of approximately 40-50° C. The mixture has been maintained on the on electric heating plate for approximately five hours. After cooling the mixture has been filtered with Watman 42 paper filter. The resulting filtrate represents the coloquintide extract containing the cucurbitacines.

The coloquintide extract, has been subjected to a second set of filtrations in order to remove the majority of occurring microbial flora. The last filtration has been carried out using 0.1 micron pore filter.

From filtrate five 20 ml aliquots have been taken and transferred in five 200 ml previously washed and sterilized round-bottomed flasks.

To each round-bottomed flask, a known amount of purified water, different from each other, has been added in order to carry out a dilution of contained extract.

Preservatives, have been added to all just mentioned solutions in order to stabilize the same. Added preservatives were methyl para-hydroxy-benzoate and propyl para-hydroxy-benzoate.

Then a quantitative analysis of cucurbitacines D, I, B and E for each solution contained in the round-bottomed flasks has been carried out.

A 7 ml aliquot has been sampled from each of 5 solutions and transferred in 5 PET containers equipped with spray nozzle. For each solution 5 containers containing the same juice and therefore cucurbitacine dose have been prepared. In total 25 containers equipped with spray nozzle have been prepared.

Treatment of Rhinosinusitis Effects.

Five patients suffering from relapsed chronic sinusitis have been selected by a family doctor. All the patients have been subjected to TAC of facial bones which detected the occurrence in everyone, in a more or less diffused way, of muco-pus in paranasal sinuses.

The first solution (more diluted one) has been delivered to the nasal fossae of each patient according to the following posology: 3 doses for naris, 2 times daily (morning and evening) for 2 days.

After the two days, that is after the fourth dose, all the patients have not displayed remarkable modification of their health conditions, i.e. no significant muco-pus depletion occurred, both from the nasal fossae and throat.

All the patients, after the end of the treatment with the first solution, have not been treated in anyway for 2 days.

At the end of the abstinence period, the second solution (solution with extract dilution lower than the first) has been delivered to the nasal sinuses of each patient, according to the same posology used in the first treatment.

At the end of the second treatment, that is after the fourth administration, all the patients have not displayed remarkable modification of their health conditions, i.e. no significant muco-pus depletion occurred, both from the nasal fossae and throat.

All the patients, after the end of the treatment with the second solution, have not been treated in anyway for 2 days.

At the end of the second abstinence period, the third solution (solution with extract dilution lower than both the first and second) has been delivered to the nasal fossae, according to the same posology used in the first treatment

At the end of the third treatment, that is after the fourth administration, all the patients have displayed meaningful changes of their health conditions, that is, they have displayed muco-pus depletion, both from the nasal fossae and throat.

All the patients, after the end of the treatment with the third solution, have not been treated in anyway for 3 days.

At the end of the third abstinence period, the fourth solution (solution with extract dilution lower than the first, second and third) has been delivered to the nasal fossae, according to the same posology used in the first treatment

At the end of the fourth treatment, that is after the fourth administration, all the patients again have displayed muco-pus depletion, both from the nasal fossae and throat.

It is pointed out that the beginning of the muco-pus depletion, as a result of the administration of the third solution, was different depending on the patient, i.e.: one patient displayed depletion already after the first administration; another patient displayed the depletion onset after the second administration and other 3 patients displayed depletion only after the third administration.

All the patients, after the evacuation of the muco-pus, experienced the disappearance of the painful symptomatology.

The fifth solution has not been administered due to the fear of significant side-effects occurrence.

As result of the carried out study, it is possible certainly to state that cucurbitacines present in coloquintide, up to a given concentration exerts no action, while, beyond said concentration, display high ability in order to evacuate muco-pus present in the paranasal sinuses formed as sinusitis consequence.

Cucurbitacine concentration ranges wherein the muco-pus evacuation has been evidenced are the following ones:
cucurbitacine D: from 7.5 μg/mL to 45.0 μg/mL, preferably from 15.5 μg/mL to 22.5 μg/mL,
cucurbitacine I: from 7.5 μg/mL to 45.0 μg/mL, preferably from 16.0 μg/mL to 23.0 μg/mL,
cucurbitacine B: from 4.5 μg/mL to 25 μg/mL, preferably from 7.6 μg/mL to 12.6 μg/mL,
cucurbitacine E from 0.5 μg/mL to 5 μg/mL, preferably from 0.9 μg/mL to 1.9 μg/mL.

### EXAMPLE 2

### Study Demonstrating that Cucurbitacines when in Contact with Nasal Mucosa, Stimulate the Leak of Some Salts Occurring in Ciliate Cells and Mucosa Connective Tissue

By the administration to nasal fossae of Coloquintide extract (containing cucurbitacines), a local depolarization of epithelial cell membrane of the nasal and paranasal mucosa is determined, which is sufficient to activate Ca⁺⁺ channels to control the potential. Ca++ ion enters the cell initiating the potential action.

As a result of membrane depolarization, the opening of K⁺ channels for the K+ to control the potential thereof occurs allowing K+ ions to leave the cytoplasm and the cell to be repolarized.

Moreover, as a result of the Ca++ entrance into the cell and increase of cytoplasmatic concentration thereof, the membrane channels are opened to Mg++, Na+ and K+ ions (channels whose opening depends on [Ca++]). It is known that calcium-dependent K+ and Na+ currents are regulated by the protein transducer calmodulin, a calcium binding protein.

By this mechanism the leak of some salts, particularly K+ and Na+, the first in higher amount, from nasal mucosa cells occurs (therefore, from same mucosa).

This process has been detected using the quantitative analysis of Ca, Mg, Na and K, present in the mucus produced by the mucosa of the paranasal sinuses and the nasal fossae in a patient suffering from acute phase rhinosinusitis.

Quantitative analysis of Ca, Mg, Na and K in the mucus collected from the patient during 24 hours before the administration of cucurbitacine containing extract and in the mucus collected from the patient during 24 hours after the administration of the same extract has been performed. Collected mucus is represented from the naturally evacuated fraction from the naris, therefore it is assumed that a portion of the produced mucus has not been collected because of outflow through the throat.

The mucus amount collected from the patient during 24 hours before the extract treatment, was equal to 3 ml, while the mucus amount collected from the same patient during 24 hours after the extract treatment was 6 ml. Already this first data are very meaningful, as, the difference of the leaked mucus amount after the treatment compared to the amount leaked before the treatment was 3 ml. This represents a greater mucus amount produced from the mucosa of the paranasal sinuses and nasal fossae, resulting from the administration of the extract.

The higher amount of collected mucus would not indicate a greater mucus amount produced by mucosa mucus producing cells, as the chemical composition thereof, particularly with reference to salts (Na, K, Mg, and Ca), is not proportional to collected mucus amount. This data would indicate that to the mucus normally produced by mucus producing cells of the paranasal sinus and nasal fossa mucosa, an amount of liquid leaked from the same mucosa as a result of the action of the administered extract, is added. This is the liquid obviously accumulated in the mucosa as a result of inflammation resulting in the edema.

From the higher amount of salts occurring in the mucus collected during 24 hours after the extract administration, and from higher amount of the collected mucus, it is apparent the mechanism through which the liquid leaked from mucosa connective tissue (hydragogue activity) is added to the mucus produced by mucus secreting cells.

The extract administration, by stimulating the leak of salts from mucosa epithelial cells (particularly Na and K), results in higher concentration thereof on the mucosa external surface (within the cavities of the paranasal sinuses and nasal fossae). This determines the equilibrium unbalance between salt concentrations inside and outside of the mucosa (concentration difference), originating an osmotic process aiming to lost equilibrium restoring. In order to reach said equilibrium, it is necessary that an amount of liquid occurring in edematous mucosa, is moved outside of the same, in this case in the cavities of the paranasal sinuses and nasal fossae.

This is the reason for the collected mucus higher amount and higher detected salt amount therein, particularly Na and K. By this mechanism it is also explained also the lesser Ca⁺⁺ amount found in mucus collected after the extract administration (calcium is removed from the mucus by mucosa epithelial cell absorption.

### EXAMPLE 3

### Higher Amount of Calcium Entered into Ciliate Cells as a Result of Cucurbitacine Stimulus Results in Change of the Motion of their Cilia

From results pointed out in example 1, it is apparent that mucosa ciliate and mucus secreting cells, as a result of the extract administration, absorb an higher amount of Ca. This Ca higher amount occurring in ciliate cells, beyond 10⁻⁶ M cytoplasmatic concentration, results in a motion modification of the cilia beat, that is, from forward to backward swimming. In fact it is known that Ca, Na, K, and Mg membrane channels act together for regulating the response duration of backward swimming according to the following mechanisms:

1. Potential controlled Ca++ channels allow the Ca++ to enter cell depolarized by the cucurbitacine induced stimulation. The Ca++ entrance into ciliate cell, triggers the onset of backward swimming response.
2. Calcium-dependent Na+ channels which are opened as calcium entrance response, extend the time the cell is depolarized and thus maintaining potential controlled Ca++ channels opened, extend the backward swimming response.
3. Potential controlled and calcium dependent K+ channels allow the cell re-polarization and, therefore, the closing of Ca++ channels and consequent forward swimming restoration.

It is apparent that a sudden motion change of ciliate cells motion, results in higher ability to carry out, according to role thereof, the transport of the mucus towards the outside of the cavities of the paranasal sinuses and nasal fossae.

### EXAMPLE 4

### Higher Liquid Amount Produced in Cavities of the Paranasal Sinuses and Nasal Fossae Results in a Decongestant and Anti-Edemigenous Effect on Nasal and Paranasal Mucosa and Higher Efficiency and Effectiveness of Ciliate Cells

As pointed out in the example, the presence of higher electrolyte amount (Na and K) in nasal fossae and, particularly, in paranasal sinuses, results in the formation of hypertonic phase in contact with the mucosa, which in turn determines the leak of liquids from the same mucosa by osmosis, which unequivocally results in decongestant and anti-edemigenous action derived from mucosa volume reduction (the higher amount of liquid accumulated in mucosa connective tissue as a result of the inflammation, moves toward outside with subsequent emptying thereof).

Due to the fact that said higher liquid amount transferred in the nasal cavities and, particularly, in the paranasal sinuses, is localized on the mucosa immediate surface and, therefore, entire thickness of ciliate cell cilia, the movement of said cilia is easier (being water less viscous and colloidal than mucus), thus facilitating the evacuation of the entire mucus amount. From this action, the restoration of the paranasal sinuses pneumatisation results.

### EXAMPLE 5

### Higher Amount of Calcium Entering Ciliate Cells as a Result of Cucurbitacine Stimulus Results in the Increase of Cilia Beat

Ciliate together with mucus secreting cells, cover the mucosa of the nasal fossae, paranasal sinuses and relative ostia.

The cilia of these cells are arranged in longitudinal rows, each subdivided in hexagonal zones, named ciliate fields.

Every cilium originates from middle of a ciliate field, that, as needed, is able to double own structural elements and cilia: new are always developed in front of preceding parts.

The cilia display a structure consisting of two middle and parallel microtubules and nine pairs of peripheral parallely arranged microtubules along the cilium axis (axoneme) according to a characteristic and constant pattern in all cell types.

The two middle microtubules end at cellular surface, while peripheral ones cross the same forming the basal corpuscle.

The cilia quickly beat in a motion opposite direction and straighten (backward beat) during a period from two to six times longer than effective beat.

While beating, the ciliates develop also a rotating movement on the left and describe narrow spirals along the motion direction.

The cilium is quite rigid during the effective beat and pushes backward the water resulting in the cell forward motion.

In backward beating, the cilium bends in higher extent and is maintained nearer to the cell body, thus moving lesser amount of water.

The cilia belonging to the same longitudinal row does not beat in phase. If we observed, we would note that some cilia are in the effective beat, while others in backward phase.

When a cilium at the extremity of a row begins the effective beat, the immediately adjacent cilium begins the same phase with little delay and so on along all the row. Like a vertically located domino row which is hit at an extremity and consequent falling movement is regularly propagated along the row like a wave. In the same way the succession of the forward and backward beats along a cilia row results in a waved motion due to phase-difference between the stroke sequences named WAVE.

This coordination type of cilia beat allows a pushing continuity to be obtained since at any moment a portion of the cilia is involved in a propulsion effective beat.

While the metazoan cilia beat in one plane, the ciliated ones have variable bending plans nearly perpendicular to the body surface allowing quick direction changes as a response to environmental stresses to be carried out. According to studies performed about this concern the site of stimuli response inducing directional changes is within the cilia or immediately nearby.

It is known that, in the cilia of ciliate cells, Mg and/or Ca associated ATP activates the dynein motion (protein binding the microtubule pair and allowing their sliding) which in turn allows the cilia beat of ciliate cells of nasal and paranasal mucosa.

It is apparent that higher Ca amount in ciliate cells, deriving from cellular ionic pump activation, as a result of cucurbitacine stimulus, unequivocally results in the increase of cilia beat and therefore higher muco-pus transport outwardly.

### EXAMPLE 6

### Cucurbitacines Exert their Surface Active Properties in Contact with Muco-Pus

In the extract, cucurbitacines are present in glycosylated form, therefore a water-soluble sugar based and a lipo-soluble sapogenine (triterpenes) based moieties are present. This structure results in saponine detergent activity (ability to attract and include lipid substances).

Saponines and, therefore sapogenines, are able to lower the surface tension in aqueous solution.

The property to lower the surface tension of a liquid facilitates different liquid surface “wettability” or miscibility, peculiarity resulting from the presence of a polar and an apolar groups. These substances, beyond a critical concentration (cmc critical micellar concentration), are arranged as supramolecular aggregates (micelles).

The micelles, formed beyond the critical concentration (cmc) represent a molecule aggregate in colloidal phase with surface-active properties. Such micelles further are characterized by the presence of water filled “aqueous pockets” due to several monomer chains which are protruding towards the outside.

The muco-pus generated in the paranasal sinuses when sinusitis occurs, is a viscous colloid secreted by mucus secreting glands of the mucosae. The mucus consists, in addition to other less important compounds, of glycosylated proteins, water dissolved mineral salts, phospholipids. These compounds, more or less interacting to each other, represent the most part of said mucus.

The mucus consists of two layers, a sol phase (peri-ciliary, mostly aqueous and ionic with low molecular weight proteins as IgAs and lysozyme) and a gel phase (epi-phase, in contact with the aerial current, insoluble and rich of high molecular weight substances). A third is inserted between said two phase which mostly consists of phospholipidic substances (phospholipids are the causative agents of mucus surface tension).

The chronic inflammatory processes of upper aerial tract result in a proliferation of caliciform mucus secreting cells with increment of the mucin and phospholipidic fraction of the mucus.

Extended thickening of mucus epi-phase (gel) and phospholipidic fraction, results in reduction of the mucus-ciliate transport (TMC) deriving from increase of both volume and mass of said phases and surface tension thereof.

Cucurbitacines, administrated as an extract by nasal spray, carry out the following actions:

Topically, as evidenced previously, they act through the stimulation (in nasal and paranasal mucosa) for the release of some electrolytes, among which the most important are Na and K, to which, by osmosis, a water flow follows (accumulated in the mucosa as a result of the inflammation) which flows into nasal fossae and paranasal sinuses. In the muco-pus presence, particularly in paranasal sinuses, higher water amount determines an increase of mucus sol phase, resulting in a first positive effect on cilia motility.

The presence of a higher electrolyte amount in the nasal fossae and paranasal sinuses determines an increase in cucurbitacine micelle formation (electrolytes are particularly important for the micelle formation). In the muco-pus, the formation of micelles (with surface active action) determines the attraction and enclosure of mucus lipid molecules (in particular phospholipids), favouring the evacuation. (the increase of mucus sol phase and micelles formation in the phospholipid phase determine a modification of aggregation state of the phospholipids and a lowering of the surface tension). Through the increase of mucus sol phase (aqueous fraction) and lowering of the surface tension at the level of the phospholipid phase, an emulsion more easily transportable towards the outside by ciliate cells of nasal and paranasal mucosa is obtained.

### EXAMPLE 7

### Cucurbitacines Exert an Indirect Anti-Inflammatory Action on Mucosa of Nasal Fossae, Paranasal Sinuses and Relative Ostia

As pointed out in example 1, the presence of higher electrolyte amount (Na and K) in nasal fossae and, particularly, paranasal sinuses, results in the formation of a hypertonic phase contacting the mucosa, which in turn results in liquid leak from said mucosa by osmosis (higher liquid amount accumulated in mucosa connective tissue as a result of inflammation, goes outside determining emptying thereof).

It is known that the inflammatory processes of nasal and paranasal mucosa determines edema, as result of the alteration of water equilibrium between the blood vessel and connective tissue contained water. The result is water build-up in connective tissue that provokes swelling up. Main process causative for the alteration of above said water equilibrium, is represented from the release histamine contained in mastocyte granules occurring in mucosa connective tissue

It is obvious that histamine, released from mucosa connective tissue, is present in therein accumulated liquid.

In the light of above, it is apparent that through the hydragogue action of cucurbitacines, the mucosa accumulated liquid flows into paranasal sinuses carrying together a meaningful portion of therein contained histamine.

It is apparent that mucosa connective tissue subtracted histamine cannot any more to carry out the action resulting in alteration of water equilibrium between the blood and connective tissue contained water, with the consequent decrease of inflammatory process.

As an evidence of the statement, quantitative analysis of histamine present in the mucus collected from a patient suffering from sinusitis during 24 hours before the administration of cucurbitacine containing extract, and in the mucus collected from the same patient during 24 hours after the administration of the same extract has been carried out. The result of quantitative analyses, shows that in the mucus collected during 24 after the extract administration the histamine was present in an amount more than twice in comparison to that in the mucus collected during 24 hours before the extract administration. Specifically in the mucus collected during 24 hours before the extract administration histamine amount was equal to 168.67 ppb, while in the mucus collected during 24 after the extract administration histamine amount was equal to 367.0 ppb.

### EXAMPLE 8

### Study on Potential Changes of Upper Respiratory Ciliate Cells as a Result of Contact with Cucurbitacins B-D-I-E

### Scientific Background

Previous experimental evidence indicates that the administration in humans, via nasal spray of Cucurbitacins B, D, I, E, present in an extract of Citrullus Colicintis, causes an electrolyte alteration in the epithelium of the nasal mucosa and paranasal sinuses, leading to a possible alteration of membrane potential, with expulsion of Na⁺ and K⁺ due to the possible absorption of Ca2⁺. A series of processes useful in the treatment of rhinosinusitis follows such primary change.

In order to ensure that such processes are originated exclusively from Cucurbitacins B, D, I, E and not from other substances present in the extract of Citrullus Colicintis, this study was carried out, based on the evaluation of the change in membrane potential of the upper respiratory ciliate cells. The study was mainly focused on defining the effects of Cucurbitacins B, D, I, E on respiratory epithelial cells, leading to their action on membrane potential and ionic currents of the cell at the base of this potential variation, and further, at determining a curve dose/response to the same Cucurbitacins.

### The Main Objectives of the Study

1) In vitro evaluation of the effect of Cucurbitacins B, D, I, E on ciliate cells of the respiratory mucosa, by measuring changes in the membrane potential;
2) In vivo evaluation of the effect of Cucurbitacins B, D, I, E on the respiratory mucosa by means of quantification of electrolytes present in the mucus produced by the same.

### Schematic Description of the Study Related to Objective 1

For the present study cellular models of commercially available respiratory epithelium cells (RPMI 2650 cells—LCG standards) were used.

The RPMI 2650 (ATCC number CCL-30) derive from human nasal septum and faithfully mimic the respiratory epithelium against the following parameters: karyotype, expression pattern of keratin polypeptides, presence of mucoid material on the cell surface.

The study was conducted primarily using the technique of molecular physiology of measurement of membrane potential and cellular currents by electrophysiological techniques.

### Electrophysiological Technique in Current-Clamp.

There have been measures of membrane potential (mV) of a single cell in the following conditions:
control saline (solution A)
with Cucurbitacine B, D, I, E, present in the aqueous extract of Coloquintide, at a concentration of 50 μg/ml (solution B)
with Cucurbitacine B, D, I, E ChromaDex pure state, in aqueous solution, at a concentration of 50 μg/ml (solution C)

### Solution A—Preparation:

ready-made solution of NaCl 0.9% P/V (9 g/L)

### Solution B—Preparation:

Citrullus Colicintis fruits, collected in a period of full maturity, were deprived of epicarp and cut into small pieces. 200 grams of the cut fruit were collected and placed in a beaker of 1000 ml, to which 500 ml of distilled water were added. The beaker with the inside mixture (water+fruit Citrullus Colicintis) was brought on electric hotplate and heated to about 40-50° C. The mixture was kept on the electric hot-plate for about five hours. After cooling, the mixture was filtered with filter paper Watman 42. The obtained filtrate represents the extract of coloquintide containing Cucurbitacins. The Citrullus Colicintis extract was subjected to a second set of filtering in order to remove most of the microbial flora. The final filtration was performed by means of a filter with porosity of 0.1 microns.

The filtrate was subjected to the quantification of Cucurbitacins present there through techniques of mass spectrometry LC-MS.

### Quantitative Analysis of Cucurbitacins B-D-I-E

### Chromatographic Conditions for Separation

TABLE 1

| | |
|---|---|
| Mobile phase A | H2O + Formic Acid 0.1% |
| Mobile phase B | CH3CN + Formic Acid 0.1% |
| Column | Ascentis C18 (2.1 × 50 − 5 um) |
| Inj | 10 μL |
| Source | ESI− |
| Method | LC_MS ESI − Ecball |

TABLE 2
GRADIENT

| Time (minutes) | % B | Flow (mL/min) |
|---|---|---|
| 0 | 5 | 0.20 |
| 3 | 5 | 0.20 |
| 30 | 90 | 0.20 |
| 38 | 90 | 0.20 |
| 39 | 5 | 0.20 |
| 43 | 5 | 0.20 |

### Samples Preparation

The samples was diluted 1:5

TABLE 3
PREPARATION OF CALIBRATION COURVE

| | |
|---|---|
| Mix Std 0.25 ppm | Punto A |
| Mix Std 0.50 ppm | Punto B |
| Mix Std 1 ppm | Punto C |
| Mix Std 5 ppm | Punto D |
| Mix Std 10 ppm | Punto E |
| Mix Std 25 ppm | Punto F |

### Samples Analysis

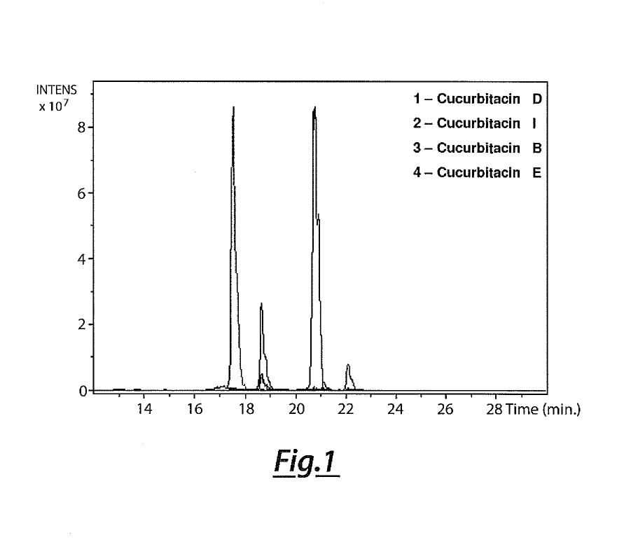

A graph is showed in FIG. 1.

Table 4 shows the results of the analysis.

TABLE 4

| Cucurbitacins | Conc. dil 1:5 | |
|---|---|---|
| Cucurbitacin B | 92.20 | μg/ml |
| Cucurbitacin D | 79.06 | μg/ml |
| Cucurbitacin I | 14.56 | μg/ml |
| Cucurbitacin E | 4.81 | μg/ml |
| Total conc. | 190.63 | μg/ml |

The concentrations were corrected for the dilution factor

The extracted solution containing Cucurbitacins B-D-I-E in the total quantity of 190.63 μg/ml, was further diluted with distilled H₂O for the dilution factor 0.262 to obtain the concentration of 50 μg/ml (solution B).

### Solution C—Preparation

The following Cucurbitacins ChromaDex, provided by the company LC Standards, were used:

| | | |
|---|---|---|
| 1. | Cucurbitacin B | |
| | Lot number | 00003916-218 |
| | Molecular formula | C₃₂H₄₆O₈ |
| | Molecular weight | 558.70 |
| | CAS Number | 6199-67-3 |
| | Chemical family | Triterpenes |
| 2. | Cucurbitacin D | |
| | Lot number | 00003914-701 |
| | Molecular formula | C₃₀H₄₄O₇ |
| | Molecular weight | 516.67 |
| | CAS Number | 3877-86-9 |
| | Chemical family | Triterpenes |
| 3. | Cucurbitacin I | |
| | Lot number | 00003915-414 |
| | Molecular formula | C₃₀H₄₂O₇ |
| | Molecular weight | 514.65 |
| | CAS Number | 2222-07-3 |
| | Chemical family | Triterpenes |
| 4. | Cucurbitacin E | |
| | Lot number | 03910-405 |
| | Molecular Formula | C₃₂H₄₄O₈ |
| | Molecular weight | 556.69 |
| | CAS NumBER | 1844-66-1 |
| | Chemical family | Triterpenes |

The Cucurbitacins B-D-I-E, in containers of 5 mg, were dissolved separately in 25 ml volumetric flasks, with the smallest amount possible of Methanol. The solutions were brought to volume with distilled H₂O. The final concentration of each Cucurbitacin in each 25 ml flask is of 200 μg/ml.

### Preparation of the Solution Containing all Four Cucurbitacins B-D-I-E:

in a 100 ml volumetric flask the four solutions containing Cucurbitacins at a concentration of 200 μg/ml were mixed, resulting in a final concentration of 50 μg/ml.

In all A, B, C solutions, preservatives were added to achieve their stabilization. The preservatives added were: propyl and methyl parahydroxybenzoate.

### Measurement of Membrane Potentials

The cells RPMI 2650 were grown cultured on plate following the directions indicated by the supplier.

At the end of the stages of cell growth, three identical subcultures were prepared and subjected to the measurement of membrane potential. First of all, for each subculture the resting potential was measured, where the values were found to be:
PLATE 1=−65 mV
PLATE 2=−65 mV
PLATE 3=−65 mV

In all three cell subcultures, with continuous measurement of the potential, A-B-C solutions were added, which were found to be in the peak in the following:
PLATE 1+SOL. A=−63 mV
PLATE 1+SOL. B=−30 mV
PLATE 1+SOL. C=−25 mV

### Conclusions

Given the results of the measurement of potential given above, there was evidence that the cells of the nasal septum (RPMI 2650) in contact with solution A (saline NaCl 0.9% P/V) showed no significant variation of the membrane potential than that measured at rest (−63 mV>−65 mV).

Measurements of cell potential after contact with solutions B and C, showed significant changes compared to the resting potential (SOL. B=−30 mV>−65 mV) and (C SOL.=−25 mV>−75 mV), such as to suggest the initiation of exchange processes of the electrolytes.

Another key element found from the results of measuring the potential after contact with the B and C solutions, is the fact that both the solution of Cucurbitacins B, D, I, E contained in the Citrullus Colicintis extract, and the Cucurbitacins B, D, I, E at pure state, showed essentially the same variation of potential (−30 mV>−25 mV), which is why we can say with absolute certainty that Cucurbitacins B, D, I, E are the only responsible for the basis of changes in action potential and hence of electrolytic consequential electrolytic exchanges.

### Schematic Description of the Study on Objective 2

In order to assess the effect on electrolytic exchanges that start after the contact of Cucurbitacins B, D, I, E with the nasal mucosa cells, two groups of three patients with pan sinusitis were treated, respectively, with the B and C solutions (G1-group: a-b-c patients; G2 group: d-e-f patients).

All patients were selected by the family doctor with a diagnosis of acute sinusitis or chronic exacerbation, with presence of muco-purulent secretion in one or more paranasal sinuses diagnosed with CT scan of the facial.

Patients in group G1 were treated with solution B prepared and used for the study of objective 1 (solution extracted from Citrullus Colicintis containing Cucurbitacins B, D, I, E at a dose of 50 μg/ml).

Patients in group G2 were treated with solution C, processed and used for the study of objective 1 (solution containing Cucurbitacins B-D-I-E at pure state, at a dose of 50 μg/ml).

The patients of both groups were treated with the following dosage:
Nasal Spray with dispenser of 60 μl
3 sprays per nostril, 2 times a day (morning and evening).

All patients, during treatment, did not smoke and did not make use of sympathomimetic drugs.

Before subjecting the patients to the treatment, they were made to collect all the possible amount of mucus which spontaneously evacuated from the nose.

After two days of treatment, all patients in both groups (G1 and G2), were made to collect all the possible mucus evacuated from the nose.

All samples of mucus collected, before and after treatment, were subjected to quantitative analysis of the following elements Ca, Na, Mg and K, with techniques of atomic absorption spectrometry. The results are the following:

### Quantity Analysis of Samples Collected Before Treatment

TABLE 5

| TREATMENT | | Ca | Na | Mg | K |
|---|---|---|---|---|---|
| GROUPS | PATIENT | mg/L | mg/L | mg/L | mg/L |
| G1 | a | 0.148 | 8.027 | 0.029 | 0.725 |
| | b | 0.155 | 8.119 | 0.035 | 0.758 |
| | c | 0.184 | 8.244 | 0.044 | 0.796 |
| G2 | d | 0.171 | 8.115 | 0.028 | 0.783 |
| | e | 0.135 | 8.321 | 0.051 | 0.715 |
| | f | 0.140 | 8.416 | 0.063 | 0.716 |

Quantity Analysis Collected after Two Days of Treatment

TABLE 6

| TREATMENT | | Ca | Na | Mg | K |
|---|---|---|---|---|---|
| GROUPS | PATIENT | mg/L | mg/L | mg/L | mg/L |
| G1 | a | 0.134 | 21.737 | 0.036 | 1.175 |
| | b | 0.130 | 21.310 | 0.033 | 1.232 |
| | c | 0.125 | 21.115 | 0.031 | 1.180 |
| G2 | d | 0.100 | 22.544 | 0.038 | 1.215 |
| | e | 0.115 | 22.899 | 0.040 | 1.311 |
| | f | 0.088 | 22.323 | 0.035 | 1.285 |

Table 7 shows the differences of the concentrations of elements Ca, Na, Mg and K found in the mucus collected before and after treatment.

TABLE 7

| GRUPPI DI | | Ca | Na | Mg | K |
|---|---|---|---|---|---|
| TRATTAMENTO | PAZIENTE | mg/L | mg/L | mg/L | mg/L |
| G1 | a | −0.014 | +13.710 | +0.007 | +0.450 |
| | b | −0.010 | +13.191 | +0.002 | +0.474 |
| | c | −0.029 | +12.871 | +0.008 | +0.384 |
| G2 | d | −0.071 | +14.429 | +0.010 | +0.532 |
| | e | −0.034 | +14.578 | +0.000 | +0.596 |
| | f | −0.052 | +14.407 | +0.008 | +0.569 |

### Conclusions

From the results given above, it is noted that the administration to patients of the solutions A and B, caused a different composition of mucus in relation to the concentration of the elements Ca, Na, Mg and K, more precisely, the concentrations of Na and K, Mg increased significantly, the concentrations of Mg remained unchanged and the concentrations of Ca decreased. This suggests that the administration of Cucurbitacins B-D-I-E, both present in the solution extracted from Citrullus Colicintis, and present in aqueous solution in the pure state, both at a concentration of 50 μg/ml, can cause a change in the composition of mucus referred to the tested electrolytes Ca, Na, Mg and K, without significant differences of concentration.

1) Pharmaceutical composition for the treatment of the effects resulting from sinusitis both acute and chronic, rhinosinusitis or rhinitis,
said pharmaceutical composition being in a pharmaceutical form suitable to the nasal administration to the upper respiratory apparatus and consisting of at least one cucurbitacin or an extract containing thereof,
together with one or more pharmaceutically acceptable adjuvant and/or excipients, said at least one cucurbitacin being present at concentration from 20 μg/ml to 120 μg/ml of total composition.

2) Pharmaceutical composition according to claim 1, wherein said at least one cucurbitacin is present at concentration from 40 μg/ml to 60 μg/ml of total composition.

3) Pharmaceutical composition according to claim 1, wherein the cucurbitacin is chosen from the group consisting of cucurbitacin D, I, B or E.

4) Pharmaceutical composition according to claim 3, wherein the concentration of cucurbitacin D is from 7.5 μg/mL to 45.0 μg/mL, of cucurbitacin I is from 7.5 μg/mL to 45.0 μg/mL, of cucurbitacin B is from 4.5 μg/mL to 25 μg/mL, of cucurbitacin E is from 0.5 μg/mL to 5 μg/mL.

5) Pharmaceutical composition according to claim 4, wherein the concentration of cucurbitacin D is from 15.5 μg/mL to 22.5 μg/mL, of cucurbitacin I is from 16.0 μg/mL to 23.0 μg/mL, of cucurbitacin B is from 7.6 μg/mL to 12.6 μg/mL, of cucurbitacin E is from 0.9 μg/mL to 1.9 μg/mL.

6) Pharmaceutical compositions according to claim 1 for use in the treatment of the effects resulting from sinusitis both acute and chronic, rhinosinusitis or rhinitis, said effects being mucosa edema of the upper respiratory apparatus, loss of the pneumatization of the paranasal sinuses, loss or reduction of the cilia motility, build-up of muco-purulent secretions in paranasal sinuses.
